(19) United States
(12) Patent Application Publication
(10) Pub. No.: US 2014/0121505 A1
(43) Pub. Date: May 1, 2014

(54) PHOTOACOUSTIC IMAGING SYSTEM AND APPARATUS, AND PROBE UNIT USED THEREWITH

(71) Applicant: FUJIFILM Corporation, Tokyo (JP)

(72) Inventor: Kaku IRISAWA, Ashigarakami-gun (JP)

(73) Assignee: FUJIFILM Corporation, Tokyo (JP)

(21) Appl. No.: 14/149,536

(22) Filed: Jan. 7, 2014

Related U.S. Application Data
(63) Continuation of application No. PCT/JP2012/004644, filed on Jul. 23, 2012.

(30) Foreign Application Priority Data
Jul. 27, 2011 (JP) 2011-164582

(51) Int. Cl. A61B 5/06; A61B 5/00

(52) U.S. Cl. CPC A61B 5/061; A61B 5/0095
USPC 600/424

(57) ABSTRACT

A photoacoustic imaging system which includes a treatment tool for surgery, a probe unit having an electroacoustic transducer section, an image generation section which generates a three-dimensional photoacoustic image, an information obtaining section which obtains information representing mutual relative positions and orientations of the treatment tool and the probe unit in a space, an image processing section which superimposes a treatment tool display on the photoacoustic image based on the information, and a control section which controls these such that the photoacoustic image superimposed with the treatment tool display is displayed on a display section in real time. When assisting in surgery, the present invention allows the surgeon to recognize the positional relationship between a treatment tool and a blood vessel in an easier and accurate way.

### TECHNICAL FIELD

The present invention relates to a photoacoustic imaging system and apparatus which generates a photoacoustic image by detecting a photoacoustic wave generated in a subject by the projection of light, and a probe unit used therewith.

### BACKGROUND ART

When a surgery is performed, sufficient care must be taken not to damage a blood vessel by a treatment tool, such as a surgical knife or the like. Heretofore, there has been a problem that it is difficult for the surgeon to confirm a blood vessel located at a place deeper than a certain depth from the surface of a living tissue of the subject with the naked eye.

Consequently, for example, Japanese Unexamined Patent Publication No. 2009-226072 discloses a method which allows a surgeon to recognize the positional relationship between a treatment tool and a blood vessel of a subject by administering an angiographic agent to the subject, then alternately projecting excitation light, which is in a specific wavelength range for causing the angiographic agent to emit light, and visible light at a predetermined time interval, generating a fluorescent image based on the excitation light and an ordinary image based on the visible light, and superimposing and displaying these images in real time, thereby reducing the risk of damaging the blood vessel.

### DISCLOSURE OF THE INVENTION

The method of Japanese Unexamined Patent Publication No. 2009-226072, however, requires the administration of an angiographic agent in advance and further requires that the administration of the angiographic agent is implemented such that the concentration of the angiographic agent in the blood is maintained constant. This may cause the operation to be complicated as a whole, although it is effective as a method for allowing the surgeon to recognize the positional relationship between the treatment tool and the blood vessel. Further, the method of Japanese Unexamined Patent Publication No. 2009-226072 may provide only two-dimensional information of the surface of a living tissue based on the fluorescent image and the ordinary image described above, so that it may sometimes be difficult for the surgeon to precisely recognize the depth from the surface of the blood vessel.

The present invention has been developed in view of the circumstances described above, and it is an object of the present invention to provide a photoacoustic imaging system and apparatus capable of allowing, in assisting in surgery, a surgeon to recognize the positional relationship between a treatment tool and a blood vessel in an easier and accurate way, and a probe unit used therewith.

In order to solve the problems described above, a photoacoustic imaging system according to the present invention is a photoacoustic imaging system in which measuring light is projected into a subject, a photoacoustic wave generated in the subject by the projection of the measuring light is detected and the photoacoustic wave is converted to an electrical signal, and a photoacoustic image is generated based on the electrical signal, the system including:

a treatment tool for surgery;

a three-dimensional image generation probe unit which includes a light projection section which projects measuring light and an electroacoustic transducer section which detects a photoacoustic wave generated in the subject by the projection of the measuring light and converts the photoacoustic wave to an electrical signal;

an image generation section which generates a three-dimensional photoacoustic image based on the electrical signal;

an information obtaining section which obtains information representing mutual relative positions and orientations of the treatment tool and the probe unit in a three-dimensional space;

an image processing section which superimposes a treatment tool display representing the position and the orientation of the treatment tool on an area of the photoacoustic image corresponding to the position where the treatment tool is located based on the information representing the positions and the orientations;

a display section which displays the photoacoustic image superimposed with the treatment tool display; and

a control section which controls the probe unit, the image generation section, the information obtaining section, and the display section such that the photoacoustic image superimposed with the treatment tool display is displayed on the display section in real time.

As used herein, the term “three-dimensional image generation probe unit” refers to a probe unit having an electroacoustic transducer section capable of receiving signals at a two-dimensional area along a living tissue surface.

In the photoacoustic imaging system according to the present invention, the electroacoustic transducer section is preferably formed of a plurality of transducer elements arranged two-dimensionally. Alternatively, the electroacoustic transducer section is preferably formed of a plurality of transducer elements arranged one-dimensionally and a scanning section which scans the plurality of transducer elements in a direction perpendicular to the arrangement direction of the plurality of transducer elements.

Further, in the photoacoustic imaging system according to the present invention, the probe unit preferably includes a first probe section having a first electroacoustic transducer section and a second probe section having a second electroacoustic transducer section, and is formed such that the first probe section and the second probe section are mutually separated, and a plane which includes a detection surface of the first electroacoustic transducer section and a plane which includes a detection surface of the second electroacoustic transducer section substantially correspond to each other.

The term that the first probe section and the second probe section are “mutually separated” refers to that the first and second probe sections are formed with an appropriate space that allows placement of a treatment tool.

The term, formed such that a plane which includes a detection surface of the first electroacoustic transducer section and a plane which includes a detection surface of the second electroacoustic transducer section “substantially correspond to each” refers to include the case where two planes which include the detection surfaces differ to the extent that allows appropriate detection of a photoacoustic wave by abutting the first probe section and the second probe section to the subject simultaneously from the viewpoint of surgical assistance, as well as the case where the two planes which includes the detection surfaces completely correspond to each other.

In the photoacoustic imaging system according to the present invention, the information obtaining section preferably obtains the information representing the positions and the orientations using a magnetic sensor or an infrared sensor. Alternatively, the image generation section preferably generates an ultrasonic image based on a reflection wave of an ultrasonic wave projected by the electroacoustic transducer section, and the information obtaining section preferably obtains the information representing the positions and the orientations by extracting an image area of the treatment tool from the ultrasonic image.

Further, the photoacoustic imaging system according to the present invention preferably further includes a blood vessel recognition section which extracts an image area representing a blood vessel in the photoacoustic image and obtains distribution information of the image area in the photoacoustic image, a distance calculation section which calculates a mutual distance between the blood vessel and the treatment tool based on the distribution information and the information representing the positions and the orientations, and a warning section which issues a warning when the distance calculated by the distance calculation section is less than or equal to a predetermined value.

A photoacoustic imaging apparatus according to the present invention is a photoacoustic imaging apparatus in which measuring light is projected into a subject, a photoacoustic wave generated in the subject by the projection of the measuring light is detected and the photoacoustic wave is converted to an electrical signal, and a photoacoustic image is generated based on the electrical signal, the apparatus including:

a three-dimensional image generation probe unit which includes a light projection section which projects measuring light and an electroacoustic transducer section which detects a photoacoustic wave generated in the subject by the projection of the measuring light and converts the photoacoustic wave to an electrical signal;

an image generation section which generates a three-dimensional photoacoustic image based on the electrical signal;

an information obtaining section which obtains information representing mutual relative positions and orientations of a treatment tool for surgery and the probe unit in a three-dimensional space;

an image processing section which superimposes a treatment tool display representing the position and the orientation of the treatment tool on an area of the photoacoustic image corresponding to the position where the treatment tool is located based on the information representing the positions and the orientations;

a display section which displays the photoacoustic image superimposed with the treatment tool display; and

a control section which controls the probe unit, the image generation section, the information obtaining section, and the display section such that the photoacoustic image superimposed with the treatment tool display is displayed on the display section in real time.

In the photoacoustic imaging apparatus according to the present invention, the electroacoustic transducer section is preferably formed of a plurality of transducer elements arranged two-dimensionally. Alternatively, the electroacoustic transducer section is preferably formed of a plurality of transducer elements arranged one-dimensionally and a scanning section which scans the plurality of transducer elements in a direction perpendicular to the arrangement direction of the plurality of transducer elements.

Further, in the photoacoustic imaging apparatus according to the present invention, the probe unit preferably includes a first probe section having a first electroacoustic transducer section and a second probe section having a second electroacoustic transducer section, and is formed such that the first probe section and the second probe section are mutually separated, and a plane which includes a detection surface of the first electroacoustic transducer section and a plane which includes a detection surface of the second electroacoustic transducer section substantially correspond to each other.

Still further, in the photoacoustic imaging apparatus according to the present invention, the information obtaining section preferably obtains the information representing the positions and the orientations using a magnetic sensor or an infrared sensor. Alternatively, the image generation section preferably generates an ultrasonic image based on a reflection wave of an ultrasonic wave projected by the electroacoustic transducer section, and the information obtaining section preferably obtains the information representing the positions and the orientations by extracting an image area of the treatment tool from the ultrasonic image.

Further, the photoacoustic imaging apparatus according to the present invention preferably further includes a blood vessel recognition section which extracts an image area representing a blood vessel in the photoacoustic image and obtains distribution information of the image area in the photoacoustic image, a distance calculation section which calculates a mutual distance between the blood vessel and the treatment tool based on the distribution information and the information representing the positions and the orientations, and a warning section which issues a warning when the distance calculated by the distance calculation section is less than or equal to a predetermined value.

A probe unit according to the present invention is a probe unit used when measuring light is projected into a subject, a photoacoustic wave generated in the subject by the projection of the measuring light is detected and the photoacoustic wave is converted to an electrical signal, and a photoacoustic image is generated based on the electrical signal, the probe unit including:

a light projection section which projects measuring light;

a first probe section having a first electroacoustic transducer section which detects a photoacoustic wave generated in the subject by the projection of the measuring light and converts the photoacoustic wave to an electrical signal; and

a second probe section having a second electroacoustic transducer section different from the first electroacoustic transducer section,

wherein the probe unit is formed such that the first probe section and the second probe section are mutually separated and a plane which includes a detection surface of the first electroacoustic transducer section and a plane which includes a detection surface of the second electroacoustic transducer section substantially correspond to each other.

The photoacoustic imaging system and apparatus according to the present invention includes, in particular, a three-dimensional image generation probe unit which includes a light projection section which projects measuring light and an electroacoustic transducer section which detects a photoacoustic wave generated in the subject by the projection of the measuring light and converts the photoacoustic wave to an electrical signal, an image generation section which generates a three-dimensional photoacoustic image based on the electrical signal, an information obtaining section which obtains information representing mutual relative positions and orientations of a treatment tool for surgery and the probe unit in a three-dimensional space, an image processing section which superimposes a treatment tool display representing the position and the orientation of the treatment tool on an area of the photoacoustic image corresponding to the position where the treatment tool is located based on the information representing the positions and the orientations, a display section which displays the photoacoustic image superimposed with the treatment tool display, and a control section which controls the probe unit, the image generation section, the information obtaining section, and the display section such that the photoacoustic image superimposed with the treatment tool display is displayed on the display section in real time. This may provide the surgeon with the positional relationship between the treatment tool and the blood vessel in an easily understandable manner through the three-dimensional image based on the photoacoustic image superimposed with the treatment tool display without requiring preprocessing, such as administering a contrast agent into a blood vessel and the like. As a result, when assisting in surgery, the surgeon is allowed to recognize the positional relationship between the treatment tool and the blood vessel easily and accurately.

The probe unit according to the present invention includes a light projection section which projects measuring light, a first probe section having a first electroacoustic transducer section which detects a photoacoustic wave generated in the subject by the projection of the measuring light and converts the photoacoustic wave to an electrical signal, and a second probe section having a second electroacoustic transducer section different from the first electroacoustic transducer section, in which the first probe section and the second probe section are formed so as to be mutually separated and such that a plane which includes a detection surface of the first electroacoustic transducer section and a plane which includes a detection surface of the second electroacoustic transducer section substantially correspond to each other, so that a surgical knife may be properly disposed within the imaging range of the photoacoustic image. This allows the surgeon to use the photoacoustic imaging system and apparatus according to the present invention to easily understand the positional relationship between the treatment tool and the blood vessel through the three-dimensional image based on the photoacoustic image superimposed with the treatment tool display without requiring preprocessing, such as administering a contrast agent into a blood vessel and the like. As a result, when assisting in surgery, the surgeon is allowed to recognize the positional relationship between the treatment tool and the blood vessel easily and accurately.

### BRIEF DESCRIPTION OF DRAWINGS

FIG. 1 is a schematic view of a photoacoustic imaging system and apparatus of a first embodiment, illustrating a configuration thereof.

FIG. 2 is a schematic view of the image generation section of the first embodiment, illustrating a configuration thereof.

FIG. 3 is a schematic view illustrating a three-dimensional photoacoustic image superimposed with a treatment tool display.

FIG. 4 is a schematic view of a photoacoustic imaging system and apparatus of a second embodiment, illustrating a configuration thereof.

FIG. 5 is a schematic view illustrating a probe unit of the second embodiment.

### BEST MODE FOR CARRYING OUT THE INVENTION

Hereinafter, embodiments of the present invention will be described with reference to the accompanying drawings, but it should be appreciated that the present invention is not limited to these embodiments. Note that each component in the drawings is not necessarily drawn to scale in order to facilitate visual recognition.

### First Embodiment

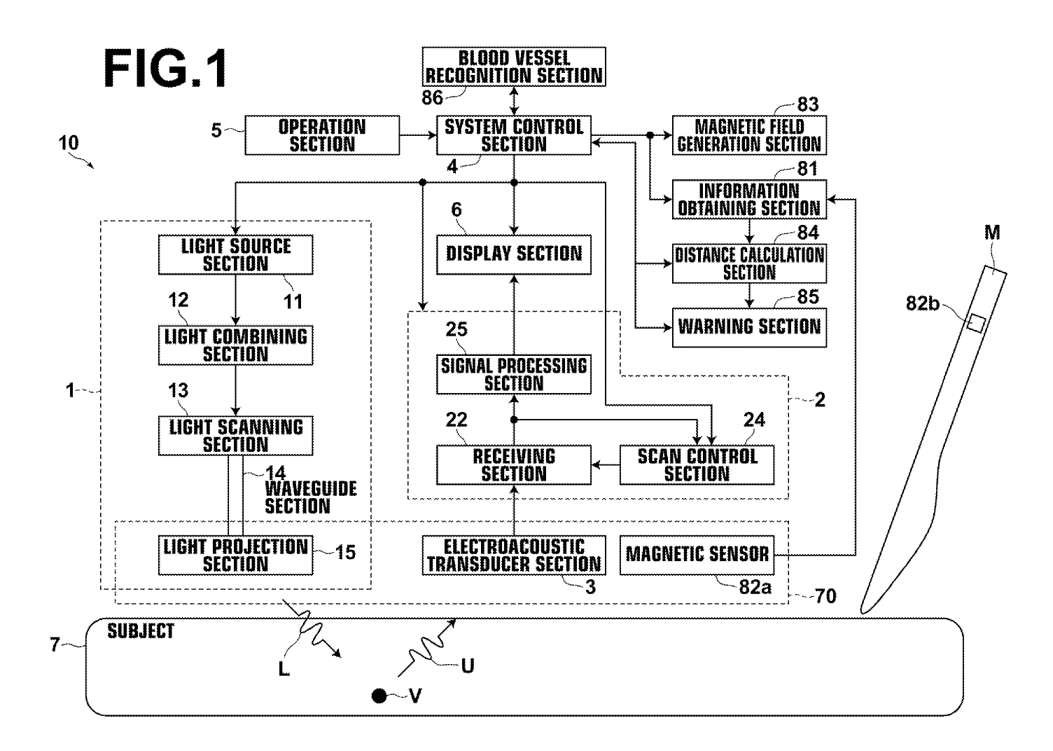

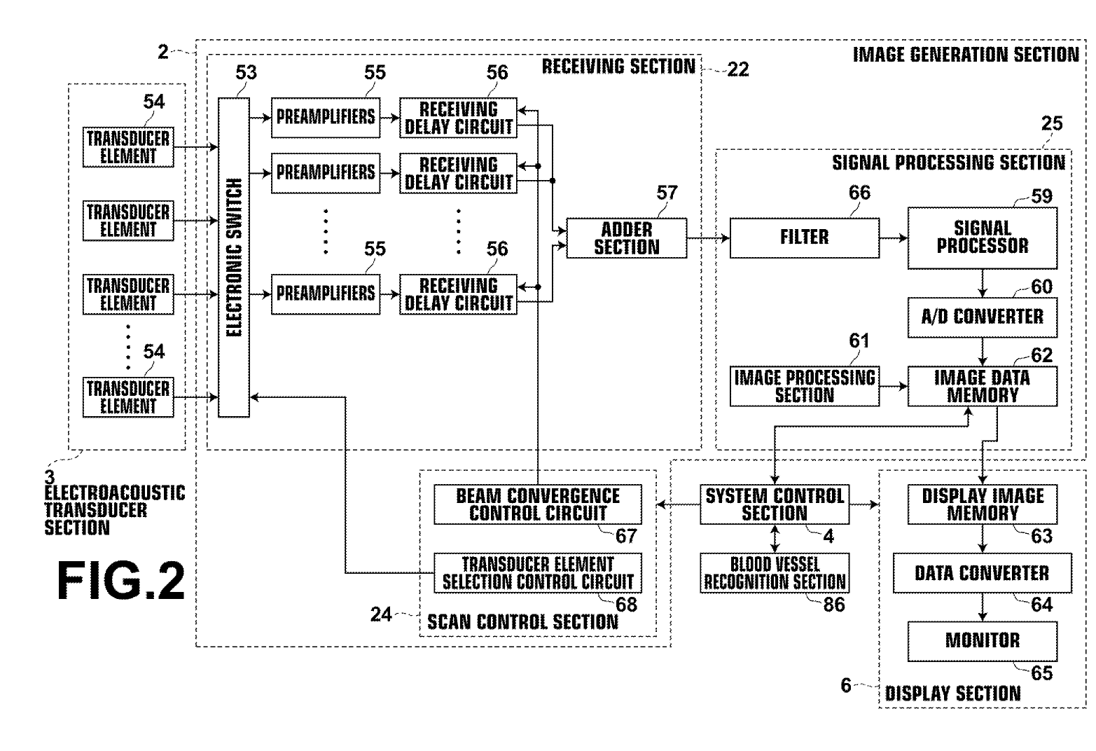

FIG. 1 is a schematic view of a photoacoustic imaging system and apparatus of the present embodiment, illustrating a configuration thereof. FIG. 2 is a schematic view of the image generation section in FIG. 1, illustrating a configuration thereof.

As illustrated in FIG. 1, the photoacoustic imaging system of the present embodiment includes a surgical knife M, as a treatment tool for surgery, and a photoacoustic imaging apparatus 10 having an information obtaining means which obtains information representing the position and the orientation of the surgical knife M in a space.

More specifically, as illustrated in FIGS. 1 and 2, the photoacoustic imaging apparatus 10 includes a light transmission section 1 which generates, as measuring light, a laser beam L which includes a particular wavelength component and projects the laser beam L onto a subject 7, an image generation section 2 which detects a photoacoustic wave U generated in the subject 7 by the projection of the laser beam L and generates photoacoustic image data of an arbitrary cross-section, an electroacoustic transducer section 3 which performs conversion between an acoustic signal and an electrical signal, a display section 6 which displays the photoacoustic image data, an operation section 5 used by the operator to enter patient information and an imaging condition of the apparatus, a magnetic sensor unit formed of a magnetic field generation section 83 and magnetic sensors 82a, 82b, an information obtaining section 81 which obtains information representing the position and the orientation of the surgical knife M in a space, a blood vessel recognition section 86 which extracts an image area representing a blood vessel from the photoacoustic image, a distance calculation section 84 which calculates a mutual distance between the blood vessel and the surgical knife M, a warning section 85 which issues a warning according to the distance described above, and a system control section 4 which performs overall control of each unit.

The probe unit 70 of the present embodiment includes the electroacoustic transducer section 3, a light projection section 15, and the magnetic sensor 82a.

The light transmission section 1 includes a light source section 11 having a plurality of light sources which outputs, for example, laser beams L of different wavelengths, a light combining section 12 which combines the later beams L of a plurality of wavelengths on the same optical axis, a multi-channel waveguide section 14 which guides the aforementioned laser beams L to a body surface of the subject 7, a light scanning section 13 which performs scanning by switching the channels used in the waveguide section 14, and the light projection section 15 from which the laser beam L supplied by the waveguide section 14 is outputted toward the imaging region of the subject 7.

The light source section 11 includes, for example, one or more light sources which generate light of predetermined wavelengths. As for the light source, a light emitting device, such as a semiconductor laser (LD), solid-state laser, or gas-laser, which generates a particular wavelength component or monochromatic light which includes the component may be used. The light source section 11 preferably outputs pulsed light, as the laser beam, having a pulse width of 1 to 100 nsec. The wavelength of the laser beam is determined as appropriate according to the light absorption properties of the measurement target substance within the subject. Although having a different optical absorption property depending on its state (oxygenated hemoglobin, deoxyhemoglobin, methemoglobin, carbon dioxide hemoglobin, or the like), the hemoglobin in a living body generally absorbs light having a wavelength of 600 nm to 1000 nm. Thus, if the measurement target is the hemoglobin in a living body (i.e., when imaging a blood vessel), the wavelength is preferably about 600 to 1000 nm. Further, the wavelength of the laser beam is preferably 700 to 1000 nm from the viewpoint that such light can reach a deep portion of the subject 7. The power of the laser beam is preferably 10 μJ/cm² to a few tens of mJ/cm² in view of the propagation losses of the laser beam and photoacoustic wave, photoacoustic conversion efficiency, detection sensitivity of current detectors, and the like. The repetition of the pulsed light output is 10 Hz or more from the viewpoint of image construction speed. Further, the laser beam may also be a pulse string in which a plurality of pulsed light is arranged side-by-side.

More specifically, when measuring, for example, a hemoglobin concentration in the subject 7, a laser beam having a pulse width of about 10 ns is formed using a Nd:YAG laser, a kind of solid-state laser, (emission wavelength: about 1000 nm) or a He—Ne gas-laser, a kind of gas-laser (emission wavelength: 633 nm). If a small light emitting device is used, such as a LD or the like, a device which uses a material, such as InGaAlP (emission wavelength: 550 to 650 nm), GaAlAs (emission wavelength: 650 to 900 nm), InGaAs or InGaAsP (emission wavelength: 900 to 2300 nm) may be used. Further, a light emitting device of InGaN which emits light with a wavelength not greater than 550 nm is becoming available in recent years. Still further, OPO (Optical Parametrical Oscillator) lasers which use a non-linear optical crystal capable of changing the wavelength may also be used.

The light combining section 12 is provided for superimposing the laser beams of different wavelengths generated from the light source section 11 on the same optical axis. Each laser beam is converted first to a parallel light beam by a collimating lens, and then the optical axis is aligned by a right angle prism or a dichroic prism. Such a configuration may provide a relatively small light combining system. Further a multi-wavelength multiplexer/de-multiplexer developed for the optical telecommunications and is available from the market may be used. In the case where a generation source, such as the OPO laser capable of continuously changing the wavelength described above or the like is used in the light source section 11, the light combining section 12 is not necessarily required.

The waveguide section 14 is provided for guiding the light outputted from the light combining section 12 to the light projection section 15. An optical fiber or a thin-film optical waveguide is used for efficient light propagation. In the present embodiment, the waveguide section 14 is formed of a plurality of optical fibers. A predetermined optical fiber is selected from the plurality of optical fibers and the laser beam is projected onto the subject 7 by the selected optical fiber. Although FIG. 1 does not clearly indicate, the optical fibers may be used in conjunction with an optical system such as an optical filter, a lens, and the like.

The light scanning section 13 supplies light while sequentially selecting a plurality of optical fibers disposed in the waveguide section 14. This allows the subject 7 to be scanned with the light.

The electroacoustic transducer section 3 is of a configuration capable of receiving signals at a two-dimensional area along a living tissue surface to allow rapid and accurate generation of a three-dimensional image. Such configuration may be realized, for example, by a plurality of transducer elements arranged two-dimensionally. It can also be realized by a plurality of transducer elements arranged one-dimensionally and a scanning section which mechanically scans the plurality of transducer elements in a direction perpendicular to the arrangement direction of the plurality of transducer elements. The transducer element 54 is a piezoelectric element formed of, for example, a piezoelectric ceramic or a polymer film, such as polyvinylidene fluoride (PVDF). The electroacoustic transducer section 3 receives a photoacoustic wave U generated in the subject 7 by the projection of light from the light projection section 15. The transducer element 54 has a function to covert the photoacoustic wave U to an electrical signal during reception. The electroacoustic transducer section 3 is constructed small and light weight, and connected to a receiving section 22 to be described later by a multi-channel cable. The electroacoustic transducer section 3 is selected from the sector scanning type, linear scanning type, and convex scanning type according to the region of diagnosis. The electroacoustic transducer section 3 may include an acoustic matching layer in order to efficiently transfer the photoacoustic wave U. Generally, the piezoelectric element material differs greatly from a living body in acoustic impedance and if the piezoelectric element material is brought into direct contact with the living body, the photoacoustic wave U cannot be transferred efficiently due to large reflection at the interface. Consequently, an acoustic matching layer having intermediate acoustic impedance is provided between the piezoelectric element material and the living body, whereby the photoacoustic wave U is transferred efficiently. Example materials of the acoustic matching layer include epoxy resin, silica glass, and the like.

The image generation section 2 of the photoacoustic imaging apparatus 10 includes a receiving section 22 which generates a receiving signal by selectively driving the plurality of transducer elements 54 constituting the electroacoustic transducer section 3 and performing in-phase addition by giving a predetermined delay time to an electrical signal from the electroacoustic transducer section 3, a scan control section 24 which controls the selective driving of the transducer elements 54 and delay time of the receiving section 22, and a signal processing section 25 which performs various kinds of processing on a receiving signal obtained from the receiving section 22. The image generation section 2 corresponds to the image generation means of the present invention.

As illustrated in FIG. 2, the receiving section 22 includes an electronic switch 53, preamplifiers 55, receiving delay circuits 56, and an adder section 57.

When receiving photoacoustic waves in the photoacoustic scanning, the electronic switch 53 sequentially selects a predetermined number of adjacent transducer elements 54. For example, if the electroacoustic transducer section 3 is formed of 192 array type transducer elements CH 1 to CH 192, such array type transducer elements are treated by the electronic switch 53 by dividing into three areas of area 0 (area of transducer elements of CH 1 to CH 64), area 1 (area of transducer elements of CH 65 to CH 128), and area 2 (area of transducer elements of CH 129 to CH 192). In this way, the array type transducer element formed of N transducer elements is treated as a section (area) of n (n<N) adjacent transducers and if imaging is performed with respect to each area, it is not necessary to connect the preamplifiers and A/D conversion boards to transducer elements of all of the channels, whereby the structure of the probe unit 70 may be simplified and cost increase may be prevented. If a plurality of optical fibers is disposed so that light is projected to each area individually, the optical power per output does not become large, which offers an advantageous effect of not requiring a high-power and expensive light source. Each electrical signal obtained by the transducer element 54 is supplied to the preamplifier 55.

The preamplifier 55 amplifies a weak electrical signal received by the transducer element 54 selected in the manner described above to ensure a sufficient S/N.

The receiving delay circuit 56 gives a delay time to the photoacoustic wave U obtained by the transducer element 54 selected by the electronic switch 53 to match the phases of photoacoustic waves U from a predetermined direction and forms a converged receiving beam.

The adder section 57 adds up electrical signals of a plurality of channels delayed by the receiving delay circuits 56 to integrate them into one receiving signal. The acoustic signals from a given depth are in-phase added by this addition and a reception convergence point is set.

The scan control section 24 includes a beam convergence control circuit 67 and a transducer element selection control circuit 68. The transducer element selection control circuit 68 supplies positional information of a predetermined number of transducer elements 54 to be selected by the electronic switch 53 during reception. In the mean time, beam convergence control circuit 67 supplies delay time information for forming a reception convergence point by the predetermined number of transducer elements 54 to the receiving delay circuits 56.

The signal processing section 25 includes a filter 66, a signal processor 59, an A/D converter 60, an image data memory 62, and an image processing section 61. The electrical signal outputted from the adder section 57 of the receiving section 22 is passed through the filter 66 to eliminate unwanted noise and a logarithmic conversion is performed on the amplitude of the received signal by the signal processor 59 to relatively emphasize a weak signal. Generally, a receiving signal from the subject 7 has amplitude with a wide dynamic range of not less than 80 dB and amplitude compression for emphasizing a weak signal is required in order to display the receiving signal on a general monitor with a dynamic range of about 23 dB. Filter 66 has band-pass characteristics with a mode in which a fundamental wave in a receiving signal is extracted and a mode in which a harmonic component is extracted. The signal processor 59 further performs envelop detection on the receiving signal subjected to the logarithmic conversion. The A/D converter 60 performs A/D conversion on the output signal from the signal processor 59 and forms photoacoustic image data of one line. The image data of one line are stored in the image data memory 62.

The image data memory 62 is a storage circuit which sequentially stores photoacoustic image data of one line generated in the manner described above. The system control section 4 reads out data of one line for a certain cross-section and required for generating a photoacoustic image of one frame stored in the image data memory 62. The system control section 4 generates photoacoustic image data of one frame of the cross-section by combining the one line data while performing spatial interpolation. Then, the system control section 4 generates three-dimensional photoacoustic image data by combining two or more photoacoustic image data of one frame changed in the position of cross-section. The system control section 4 stores the three-dimensional photoacoustic image data in the image data memory 62.

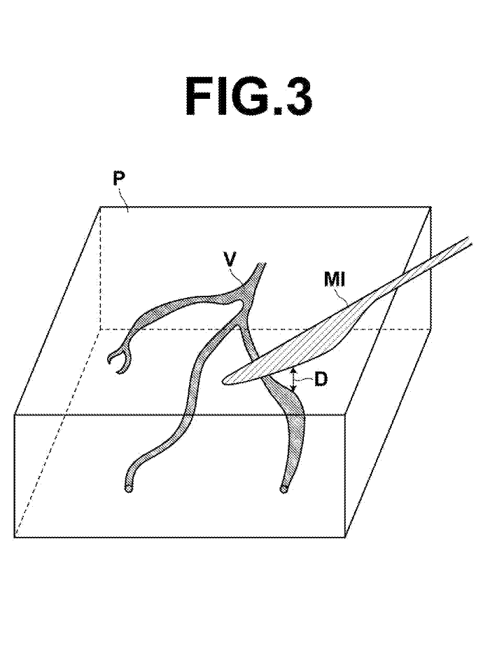

The image processing section 61 reads out the three-dimensional image data from the image data memory 62 and performs processing on a photoacoustic image P which is based on the three-dimensional image data. More specifically, based on information representing the position and the orientation of the surgical knife M obtained by an information obtaining section 81, to be described later, the image processing section 61 superimposes a surgical knife display MI (treatment tool display) on an area of the photoacoustic image P corresponding to the position where the surgical knife M is located, as illustrated in FIG. 3. The data of the photoacoustic image P superimposed with the surgical knife display MI are stored again in the image data memory 62.

The display section 6 includes a display image memory 63, a photoacoustic image data converter 64, and a monitor 65. The display image memory 63 is a buffer memory which reads out three-dimensional photoacoustic image data (i.e., the data of the photoacoustic image P superimposed with the surgical knife display MI) to be displayed on the monitor 65 from the image data memory 62 and temporarily stores them. The photoacoustic image data converter 64 performs D/A conversion and TV format conversion on the three-dimensional photoacoustic image data stored in the display image memory 63 and the output is displayed on the monitor 65. The display section 6 corresponds to the display means of the present invention.

The operation section 5 includes a keyboard, trackball, mouse, and the like on the operation panel and used by the operator of the apparatus to input required information, such as the patient information, imaging conditions of the apparatus, cross-section to be displayed, and the like.

The magnetic sensors 82a, 82b and magnetic field generation section 83 constitute a three-dimensional magnetic sensor unit for obtaining information representing mutual relative positions and orientations of the probe unit 70 and the surgical knife M in a three-dimensional space. The three-dimensional magnetic sensor unit may obtain positional coordinates (x, y, z) of the magnetic sensors 82a, 82b relative to the magnetic field generation section 83 in a space of pulsed magnetic field formed by the magnetic field generation section 83 and orientation information of the magnetic sensors 82a, 82b (information of angles (α, β, γ)). For example, the orientation information of the probe unit 70 is, for example, information related to the state of the probe unit 70 in a xyz axis space with the origin at the magnetic field generation section 83 and includes, in particular, information of inclination and rotation from the reference state in the space. There is not any specific restriction on the place where the magnetic field generation section 83 is disposed and the magnetic field generation section 83 may be disposed at any place as long as the operation range of the probe unit 70 is included in the magnetic field space formed by the magnetic field generation section 83. Each of the magnetic sensors 82a and 82b may be formed of a plurality of magnetic sensors for obtaining the information representing the positions and the orientations of the probe unit 70 and the surgical knife M described above.

The information obtaining section 81 uses the three-dimensional magnetic sensor unit and receives the information representing the positions and the orientations of the probe unit 70 and the surgical knife M in a space from each of the magnetic sensors 82a, 82b in real time. That is, information representing the position and the orientation of the probe unit 70 with respect to the magnetic field generation section 83 may be obtained from the magnetic sensor 82a while information representing the position and the orientation of the surgical knife M with respect to the magnetic field generation section 83 may be obtained from the magnetic sensor 82b. The three-dimensional magnetic sensor unit and information obtaining section 81 constitute the information obtaining means in the present invention. The information representing the position and the orientation of the probe unit 70 with respect to the magnetic field generation section 83 and the information representing the position and the orientation of the surgical knife M with respect to the magnetic field generation section 83 are sent to the distance calculation section 84.

The blood vessel recognition section 86 reads the three-dimensional photoacoustic image data generated by the image generation section 2 and extracts an image area representing a blood vessel from the photoacoustic image and obtains distribution information of the image area in the photoacoustic image. In the photoacoustic image, the image is generated using the photoacoustic effect of blood vessel and an image area representing a blood vessel may be extracted easily by any known method. The blood vessel recognition section 86 corresponds to the blood vessel recognition means of the present invention.

Based on the information representing the positions and the orientations of the probe unit 70 and the surgical knife M in a space with respect to the magnetic field generation section 83 transmitted from the information obtaining section 81, the distance calculation section 84 calculates information representing mutual relative positions and orientations of the probe unit 70 and the surgical knife M. Further, based on the positional relationship of the probe unit 70 and the surgical knife M with respect to the imaging area, as well as the information representing the positions and the orientations described above, the distance calculation section 84 calculates a distance D between the blood vessel V and the surgical knife display MI in a virtual space of the photoacoustic image (FIG. 3). The distance calculation section 84 corresponds to the distance calculation means in the present invention. The term “distance” as used herein refers to an index for ensuring a treatment tool, such as a surgical knife and the like, to be located within a range not to damage a blood vessel. The determination as to which parts of the blood vessel V and the surgical knife display MI are to be used for the calculation of the “distance” may be made as appropriate. As for the reference point of the blood vessel V, a portion of the extracted blood vessel closest to the surgical knife display MI or a portion of the blood vessel having a predetermined size and being closest to the surgical knife display MI may be cited as examples. In the mean time, as for the reference point of the surgical knife display, a portion of the surgical knife display MI closest to the blood vessel V or a point arbitrarily set on the surgical knife display MI may be cited. In the case where a portion of the extracted blood vessel V closest to the surgical knife display MI is used as the reference point of the blood vessel V and a portion of the surgical knife display MI closest to the blood vessel V is used as the reference point of the surgical knife display MI, the shortest distance between the extracted blood vessel V and the surgical knife display MI may be obtained. The distance D calculated by the distance calculation section 84 is transmitted to the warning section 85. The distance calculation section 84 corresponds to the distance calculation means in the present invention.

The warning section 85 is provided to issue a warning when the distance ID transmitted from the distance calculation section 84 is less than or equal to a predetermined value. The predetermined value is set, for example, by the operation section 5 in advance. The warning is implemented by issuing a warning sound or displaying a warning screen on the display section 6. The warning section 85 corresponds to the warning means in the present invention.

The system control section 4 controls the entire system such that the photoacoustic image P superimposed with the surgical knife display MI is displayed on the display section 6 in real time. The system control section 4 corresponds to the system control means in the present invention. In order to properly assisting in surgery, the display of the photoacoustic image is preferably performed at an image construction speed of 10 frames/sec or greater and more preferably 15 to 60 frames/sec or greater. Consequently, the system control section 4 projects the laser beam L at a repetition frequency of not less than 10 Hz and more preferably not less than 15 to 60 Hz and controls the entire system in synchronization with the projection of the laser beam L. More specifically, for example, the system control section 4 controls the probe unit 70 to receive a photoacoustic wave and/or an ultrasonic wave, the image generation section 2 to generate a photoacoustic image and/or an ultrasonic image, the three-dimensional magnetic sensor unit and information obtaining section 81 to obtain the information representing the mutual relative positions and the orientations of the probe unit 70 and the surgical knife M, and the display section 6 to display the photoacoustic image and/or the ultrasonic image in synchronization with the projection of the laser beam L.

As described above, the photoacoustic imaging system and apparatus of the present embodiment includes: in particular, a three-dimensional image generation probe unit having a light projection section which projects measuring light and an electroacoustic transducer section which detects a photoacoustic wave generated in the subject by the projection of the measuring light and converts the photoacoustic wave to an electrical signal; an image generation means which generates a three-dimensional photoacoustic image based on the electrical signal; an information obtaining means which obtains information representing mutual relative positions and orientations of the treatment tool and the probe unit in a three-dimensional space; an image processing means which superimposes a treatment tool display representing the position and the orientation of the treatment tool on an area of the photoacoustic image corresponding to the position where the treatment tool is located based on the information representing the positions and the orientations; a display means which displays the photoacoustic image superimposed with the treatment tool display; and a control means which controls the probe unit, the image generation means, the information obtaining means, and display means such that the photoacoustic image superimposed with the treatment tool display is displayed on the display means in real time. This may provide surgeon with the positional relationship between a treatment tool and a blood vessel in an easily understandable manner through the three-dimensional image based on the photoacoustic image superimposed with the treatment tool display without requiring preprocessing, such as administering a contrast agent into a blood vessel and the like. As a result, when assisting in surgery, the surgeon is allowed to recognize the positional relationship between the treatment tool and the blood vessel easily and accurately.

### <Design Change>

In the first embodiment, the description has been made that the information obtaining means obtains the information representing the positions and the orientations described above using magnetic sensors, but infrared sensors may be used instead of the magnetic sensors.

Further, if the image generation means is configured to generate an ultrasonic image based on a reflection wave of an ultrasonic wave projected by the electroacoustic transducer section described above, the information obtaining means may be configured to obtain the information representing the positions and the orientations described above by extracting an image area representing the treatment tool from the ultrasonic image. More specifically, for example, an arrangement may be adopted in which a photoacoustic image and an ultrasonic image are generated alternately by 1/60 and from the shadow of a treatment tool captured in the ultrasonic image, information representing the spatial position and the orientation of the treatment tool is extracted. Otherwise, if the ultrasonic image is captured simultaneously, simple superimposition of the ultrasonic image and the photoacoustic image after positional alignment may provide an advantageous effect that the positional relationship between a treatment tool and a blood vessel is understood. In the case where the information representing the position and the orientation of the treatment tool is obtained using the ultrasonic image in the manner described above, the existing probe unit and image generation means may be used, so that the cost for providing the magnetic sensors and the like may be saved.

### Second Embodiment

A photoacoustic imaging system and apparatus according to a second embodiment will now be described. The photoacoustic imaging system and apparatus of the present embodiment differs from the photoacoustic imaging system and apparatus of the first embodiment in the structure of the probe unit. Therefore, the components identical to those of the first embodiment are given the same reference symbols and will not be elaborated upon further here unless otherwise specifically required.

The photoacoustic imaging system of the present embodiment includes a surgical knife M, as a treatment tool for surgery, and a photoacoustic imaging apparatus 10 having an information obtaining means which obtains information representing the position and the orientation of the surgical knife M in a space.

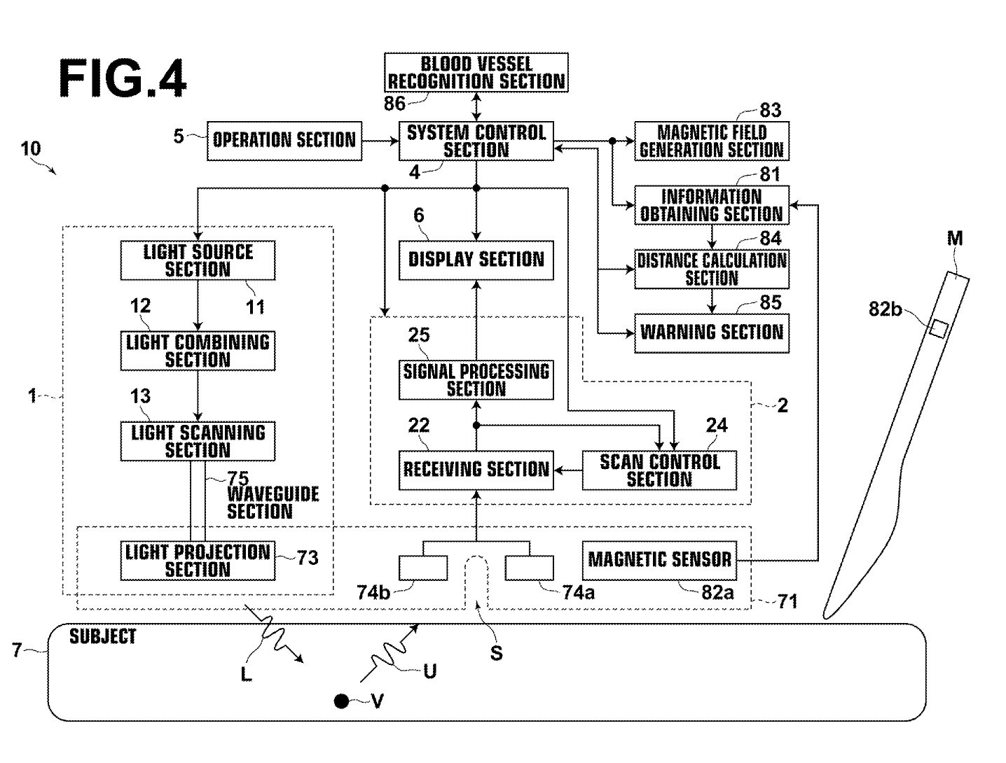

More specifically, as illustrated in FIG. 4, the photoacoustic imaging apparatus 10 includes a light transmission section 1 which generates, as measuring light, a laser beam L which includes a particular wavelength component and projects the laser beam L onto a subject 7, an image generation section 2 which detects a photoacoustic wave U generated in the subject 7 by the projection of the laser beam L and generates photoacoustic image data of an arbitrary cross-section, an electroacoustic transducer section 74a, 74b which perform conversion between an acoustic signal and an electrical signal, a display section 6 which displays the photoacoustic image data, an operation section 5 used by the operator to enter patient information and an imaging condition of the apparatus, a magnetic sensor unit formed of a magnetic field generation section 83 and magnetic sensors 82a, 82b, an information obtaining section 81 which obtains information representing the position and the orientation of the surgical knife M in a space, a blood vessel recognition section 86 which extracts an image area representing a blood vessel in the photoacoustic image, a distance calculation section 84 which calculates a mutual distance between the blood vessel and the surgical knife M, a warning section 85 which issues a warning according to the distance described above, and a system control section 4 which performs overall control of each unit.

In the present embodiment, the probe unit 71 includes a light projection section 73 that projects measuring light, a first probe section 72a having a first electroacoustic transducer section 74a which detects a photoacoustic wave generated in a subject by the projection of the measuring light and converts the photoacoustic wave to an electrical signal, a second probe section 72b having a second electroacoustic transducer section 74b which is different from the first electroacoustic transducer section 74a, and a magnetic sensor (not shown), and is formed such that the first probe section 72a and the second probe section 72b are mutually separated, and a plane which includes the detection surface 76a of the first electroacoustic transducer section 74a (bottom surface of the electroacoustic transducer section) and a plane which includes the detection surface 76b of the second electroacoustic transducer section 74b substantially correspond to each other.

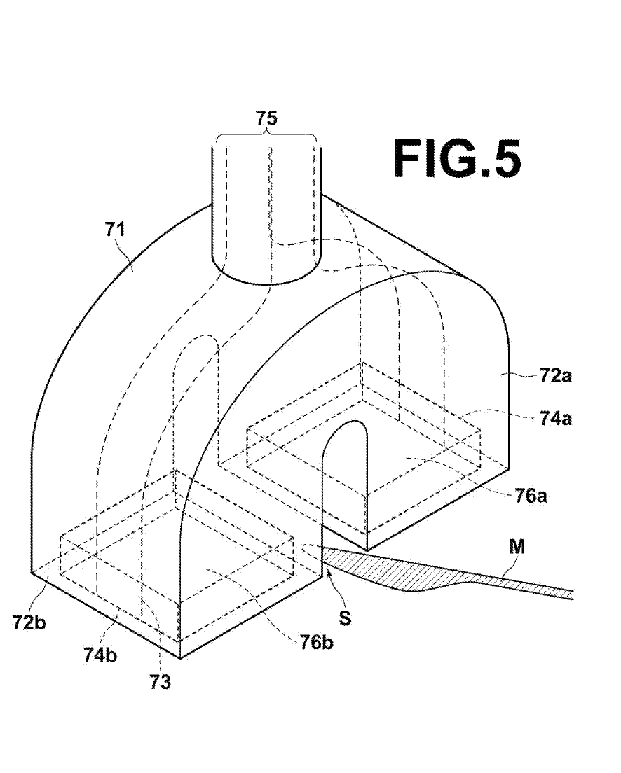

That is, the probe unit 71 illustrated in FIG. 5 has a forked structure in which the first probe section 72a and the second probe section 72b are mutually separated to allow the surgical knife M to be inserted into the space S between the first probe section 72a and the second probe section 72b. The width of the space S is preferably 1 to 10 mm.

The light projection section 73 is, for example, a tip portion of a waveguide section 75, such as an optical fiber, and is provided for guiding the laser beam L around each of the two electroacoustic transducer sections. In FIG. 4, only some of the waveguide sections are illustrated for convenience. The waveguide section 75 is identical to the waveguide section 14 in the first embodiment.

Each of the first probe section 72a and the second probe section 72b functions as a probe for performing photoacoustic imaging. As the first probe section 72a and the second probe section 72b are abutted to the subject simultaneously, they are constructed such that the plane which includes the detection surface 76a of the first electroacoustic transducer section 74a and the plane which includes the detection surface 76b of the second electroacoustic transducer section 74b substantially correspond to each other. When the probe unit 71 is abutted to a subject, this allows the two detection surfaces 76a and 76b to be disposed at the same height from the surface of a living tissue, whereby variations in detection signal may be reduced.

As each of the first electroacoustic transducer section 74a and the second electroacoustic transducer section 74b can be regarded as the electroacoustic transducer section 3 in the first embodiment divided into two regions, the way they are driven, the material, and the like are substantially identical to those of the electroacoustic transducer section 3. For example, data of one photoacoustic image are generated by combining the signal detected by each of the first electroacoustic transducer section 74a and the second electroacoustic transducer section 74b and stored in the image data memory 62. Here, with respect to the photoacoustic image data directly beneath the space S, the obtainable signal is reduced by the amount corresponding to the space S, but it is possible to generate photoacoustic image data directly beneath the space S. Generally, photoacoustic image data for one line are generated using detection data of 64 channels, and even if a space S of 1 to 10 mm (which is the length corresponding to about 4 to 33 channels) exists, the photoacoustic image data may be constructed using the detection data of the remaining channels of about 31 to 60. In the aforementioned case, the intensity of the signal in-phase added by the adder section 57 drops as a result of the reduced amount of obtainable signal. Therefore, additional signal processing, such as emphasis processing and the like, may be performed on the aforementioned in-phase added signal as required in the present embodiment.

The processing for superimposing an image and displaying, processing for extracting a blood vessel, processing for calculating a distance between the blood vessel and the surgical knife, processing for issuing a warning, and the like which follow are identical to those of the first embodiment.

In the present embodiment, the first probe section 72a and the second probe section 72b are mutually separated to form a forked structure to allow the surgical knife M to be inserted into the space S between them, so that a surgical knife may be properly disposed within the imaging range of the photoacoustic image. This may improve the accuracy in surgical assistance using the photoacoustic imaging system and apparatus of the present invention. As a result, when assisting in surgery, the surgeon is allowed to recognize the positional relationship between the treatment tool and the blood vessel easily and accurately.

What is claimed is:

1. A photoacoustic imaging system in which measuring light is projected into a subject, a photoacoustic wave generated in the subject by the projection of the measuring light is detected and the photoacoustic wave is converted to an electrical signal, and a photoacoustic image is generated based on the electrical signal, the system comprising:
a treatment tool for surgery;
a three-dimensional image generation probe unit which includes a light projection section which projects measuring light and an electroacoustic transducer section which detects a photoacoustic wave generated in the subject by the projection of the measuring light and converts the photoacoustic wave to an electrical signal;
an image generation section which generates a three-dimensional photoacoustic image based on the electrical signal;
an information obtaining section which obtains information representing mutual relative positions and orientations of the treatment tool and the probe unit in a three-dimensional space;
an image processing section which superimposes a treatment tool display representing the position and the orientation of the treatment tool on an area of the photoacoustic image corresponding to the position where the treatment tool is located based on the information representing the positions and the orientations;
a display section which displays the photoacoustic image superimposed with the treatment tool display; and
a control section which controls the probe unit, the image generation section, the information obtaining section, and the display section such that the photoacoustic image superimposed with the treatment tool display is displayed on the display section in real time.

2. The photoacoustic imaging system of claim 1, wherein the electroacoustic transducer section is formed of a plurality of transducer elements arranged two-dimensionally.

3. The photoacoustic imaging system of claim 1, wherein the electroacoustic transducer section is formed of a plurality of transducer elements arranged one-dimensionally and a scanning section which scans the plurality of transducer elements in a direction perpendicular to the arrangement direction of the plurality of transducer elements.

4. The photoacoustic imaging system of claim 1, wherein the probe unit includes a first probe section having a first electroacoustic transducer section and a second probe section having a second electroacoustic transducer section, and is formed such that the first probe section and the second probe section are mutually separated, and a plane which includes a detection surface of the first electroacoustic transducer section and a plane which includes a detection surface of the second electroacoustic transducer section substantially correspond to each other.

5. The photoacoustic imaging system of claim 1, wherein the information obtaining section obtains the information representing the positions and the orientations using a magnetic sensor or an infrared sensor.

6. The photoacoustic imaging system of claim 1, wherein:
the image generation section generates an ultrasonic image based on a reflection wave of an ultrasonic wave projected by the electroacoustic transducer section; and
the information obtaining section obtains the information representing the positions and the orientations by extracting an image area of the treatment tool from the ultrasonic image.

7. The photoacoustic imaging system of claim 1, further comprising:
a blood vessel recognition section which extracts an image area representing a blood vessel in the photoacoustic image and obtains distribution information of the image area in the photoacoustic image;
a distance calculation section which calculates a mutual distance between the blood vessel and the treatment tool based on the distribution information and the information representing the positions and the orientations; and
a warning section which issues a warning when the distance calculated by the distance calculation section is less than or equal to a predetermined value.

8. A photoacoustic imaging apparatus in which measuring light is projected into a subject, a photoacoustic wave generated in the subject by the projection of the measuring light is detected and the photoacoustic wave is converted to an electrical signal, and a photoacoustic image is generated based on the electrical signal, the apparatus comprising:
a three-dimensional image generation probe unit which includes a light projection section which projects measuring light and an electroacoustic transducer section which detects a photoacoustic wave generated in the subject by the projection of the measuring light and converts the photoacoustic wave to an electrical signal;
an image generation section which generates a three-dimensional photoacoustic image based on the electrical signal;
an information obtaining section which obtains information representing mutual relative positions and orientations of a treatment tool for surgery and the probe unit in a three-dimensional space;
an image processing section which superimposes a treatment tool display representing the position and the orientation of the treatment tool on an area of the photoacoustic image corresponding to the position where the treatment tool is located based on the information representing the positions and the orientations;
a display section which displays the photoacoustic image superimposed with the treatment tool display; and
a control section which controls the probe unit, the image generation section, the information obtaining section, and the display section such that the photoacoustic image superimposed with the treatment tool display is displayed on the display section in real time.

9. The photoacoustic imaging apparatus of claim 8, wherein the electroacoustic transducer section is formed of a plurality of transducer elements arranged two-dimensionally.

10. The photoacoustic imaging apparatus of claim 8, wherein the electroacoustic transducer section is formed of a plurality of transducer elements arranged one-dimensionally and a scanning section which scans the plurality of transducer elements in a direction perpendicular to the arrangement direction of the plurality of transducer elements.

11. The photoacoustic imaging apparatus of claim 8, wherein the probe unit includes a first probe section having a first electroacoustic transducer section and a second probe section having a second electroacoustic transducer section, and is formed such that the first probe section and the second probe section are mutually separated, and a plane which includes a detection surface of the first electroacoustic transducer section and a plane which includes a detection surface of the second electroacoustic transducer section substantially correspond to each other.

12. The photoacoustic imaging apparatus of claim 8, wherein the information obtaining section obtains the information representing the positions and the orientations using a magnetic sensor or an infrared sensor.

13. The photoacoustic imaging apparatus of claim 8, wherein:
the image generation section generates an ultrasonic image based on a reflection wave of an ultrasonic wave projected by the electroacoustic transducer section; and
the information obtaining section obtains the information representing the positions and the orientations by extracting an image area of the treatment tool from the ultrasonic image.

14. The photoacoustic imaging apparatus of claim 8, further comprising:
a blood vessel recognition section which extracts an image area representing a blood vessel in the photoacoustic image and obtains distribution information of the image area in the photoacoustic image;
a distance calculation section which calculates a mutual distance between the blood vessel and the treatment tool based on the distribution information and the information representing the positions and the orientations; and
a warning section which issues a warning when the distance calculated by the distance calculation section is less than or equal to a predetermined value.

15. A probe unit used when measuring light is projected into a subject, a photoacoustic wave generated in the subject by the projection of the measuring light is detected and the photoacoustic wave is converted to an electrical signal, and a photoacoustic image is generated based on the electrical signal, the probe unit comprising:
a light projection section which projects measuring light;
a first probe section having a first electroacoustic transducer section which detects a photoacoustic wave generated in the subject by the projection of the measuring light and converts the photoacoustic wave to an electrical signal; and
a second probe section having a second electroacoustic transducer section different from the first electroacoustic transducer section,
wherein the probe unit is formed such that the first probe section and the second probe section are mutually separated and a plane which includes a detection surface of the first electroacoustic transducer section and a plane which includes a detection surface of the second electroacoustic transducer section substantially correspond to each other.

16. The probe unit of claim 15, wherein each of the first electroacoustic transducer section and the second electroacoustic transducer section is formed of a plurality of transducer elements arranged two-dimensionally.

17. The probe unit of claim 15, wherein each of the first electroacoustic transducer section and the second electroacoustic transducer section is formed of a plurality of transducer elements arranged one-dimensionally and a scanning section which scans the plurality of transducer elements in a direction perpendicular to the arrangement direction of the plurality of transducer elements.

18. The probe unit of claim 15, wherein the probe unit comprises a magnetic sensor or an infrared sensor.

19. The probe unit of claim 15, wherein the light projection section guides the measuring light around each of the first electroacoustic transducer section and the second electroacoustic transducer section.

20. The probe unit of claim 15, wherein the width of the space between the first probe section and the second probe section is 1 to 10 mm.
